(19) United States
(12) Patent Application Publication
(10) Pub. No.: US 2008/0147008 A1
(43) Pub. Date: Jun. 19, 2008

(54) OPTICAL DETECTION OF MEDICAL PUMP ROTOR POSITION

(75) Inventors: Thomas G. Lewis, O'Fallon, IL (US); Mark A. Davis, O'Fallon, MO (US)

Correspondence Address: TYCO HEALTHCARE - EDWARD S. JARMOLOWICZ, 15 HAMPSHIRE STREET, MANSFIELD, MA, 02048, US

(73) Assignee: TYCO HEALTHCARE GROUP LP, Mansfield, MA (US)

(21) Appl. No.: 11/611,693

(22) Filed: Dec. 15, 2006

(51) Int. Cl. A61M 5/145

(52) U.S. Cl. 604/155

(57) ABSTRACT

Rotation of a rotatalbe assembly of a medical pumping apparatus such as a pump rotor of a rotary peristaltic pump is monitored via electromagnetic radiation (e.g., infrared radiation). The rotatable assembly is formed such that it has portions that are reflective and non-reflective with respect to the electromagnetic radiation. A electromagnetic radiation emitter-detector pair is positioned such that as the rotatable assembly rotates, the reflective and non-reflective portions alternately pass through the electromagnetic radiation emitted by the emitter. The detector receives electromagnetic radiation reflected by the reflective portions, but the non-reflective portions abosorb or disperse the radiation such that it is not received by the detector. An output circuit provides a signal indicating the position of the rotatable assembly as a function of the electromagnetic radiation received by the detector.

### BACKGROUND

Detecting the position and rotation of a pump rotor of a pumping apparatus (e.g. a rotary style peristaltic pump) allows the pumping apparatus to determine a rate of fluid delivery as well as some error conditions of the pumping apparatus. Typically, an electric motor drives the pump rotor such that pump rotor rotational speed and position can be estimated by monitoring a current and/or a voltage of the electric motor. However, some pumping applications, such as pumps which deliver medical fluids to a patient, require greater accuracy. One approach is to position magnets in a surface of the pump rotor and detect rotation of the pump rotor via a nearby Hall Effect sensor. This approach requires relatively expensive magnets and Hall Effect sensors and is necessarily adversely affected by other magnetic fields. Additionally, Hall Effect sensors produce partial sinusoid detection signals, the transitions times of which limit the amount of time that the detection signals spend at higher magnitudes, thus increasing the likelihood of detection signal inaccuracies. For example, if the detection signal is being digitally sampled and compared to a threshold, the sample may miss the peak of the sinusoid signal causing the system to miss detecting a magnet passing by the sensor. This effect would become more likely for relatively low sample rates and relatively high rotational speeds.

### SUMMARY

A medical pump embodying aspects of the invention provides a more cost-effective monitoring approach that is not adversely affected by magnetic fields. In an aspect of the invention, a portion of the pump rotor reflects electromagnetic radiation from an emitter. As the pump rotor rotates, a detector receives the reflected electromagnetic radiation. By monitoring the received electromagnetic radiation, the pump determines pump rotor position and rotation.

One aspect of the invention is directed to a medical pumping apparatus having a motor for driving a pump rotor and an electromagnetic radiation emitter-detector pair. The pump rotor includes a surface with a reflective portion for reflecting electromagnetic radiation and a non-reflective portion which does not reflect electromagnetic radiation. The emitter is positioned to emit electromagnetic radiation sequentially on the reflective portion and the non-reflective portion of the surface of the pump rotor as the pump rotor rotates. The detector receives electromagnetic radiation reflected by the reflective portion of the surface and provides a detection signal indicative of the received electromagnetic radiation for monitoring a position of the pump rotor.

Another aspect of the invention includes a pump rotor for a medical pumping apparatus comprising a motor, an emitter, and a detector. A motor shaft engages and supports the pump rotor relative to the medical pumping apparatus. A surface of the pump rotor includes a reflective portion for reflecting electromagnetic radiation and a non-reflective portion that does not reflect electromagnetic radiation. The emitter of the medical pumping apparatus emits electromagnetic radiation on the surface of the pump rotor, and is positioned to emit electromagnetic radiation sequentially on the reflective portion and the non-reflective portion of the surface of the pump rotor as the pump rotor rotates. The detector is positioned to receive electromagnetic radiation reflected by the pump rotor.

A method for detecting the rotation of a pump rotor in a medical pumping apparatus embodies yet another aspect of the invention. A surface having a reflective portion for reflecting electromagnetic radiation and a non-reflective portion that does not reflect electromagnetic radiation is provided on the pump rotor. Electromagnetic radiation is emitted on the surface of the pump rotor such that as the pump rotor rotates, the electromagnetic radiation sequentially interacts with the reflective portion and the non-reflective portion of the surface. A detector receives electromagnetic radiation reflected by the pump rotor and provides a detection signal in response thereto.

This summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### BRIEF DESCRIPTION OF THE DRAWINGS

FIG. 1 is a perspective view of a pumping apparatus.

FIG. 2 is perspective, exploded, and partially schematic view of a pumping apparatus illustrating one embodiment having part of a housing of the pumping apparatus cut away wherein an emitter detector pair is mounted in the housing.

FIG. 3 is a perspective, exploded, and partially schematic view of a pumping apparatus illustrating one embodiment having part of a housing of the pumping apparatus cut away wherein an emitter-detector pair is mounted behind a transmissive window of the housing.

FIG. 4 is a partially exploded view of a pump rotor according to another embodiment.

FIG. 5 is a partial perspective view of a pumping apparatus according to another embodiment illustrating a disk mounted on a shaft operatively connected to a motor of the pumping apparatus and monitored by an emitter detector pair.

FIG. 6A is a partial side view of a pumping apparatus illustrating one embodiment having a disk on a shaft of a motor of the pumping apparatus monitored by an emitter detector pair.

FIG. 6B is a partial end view of the pumping apparatus of FIG. 5A illustrating the disk and emitter detector pair.

FIG. 7 is a perspective view of a pumping apparatus according to another embodiment illustrating a disk with notches mounted on a shaft operatively connected to a motor of the pumping apparatus and monitored by an emitter detector pair.

Corresponding reference characters indicate corresponding parts throughout the drawings.

### DETAILED DESCRIPTION

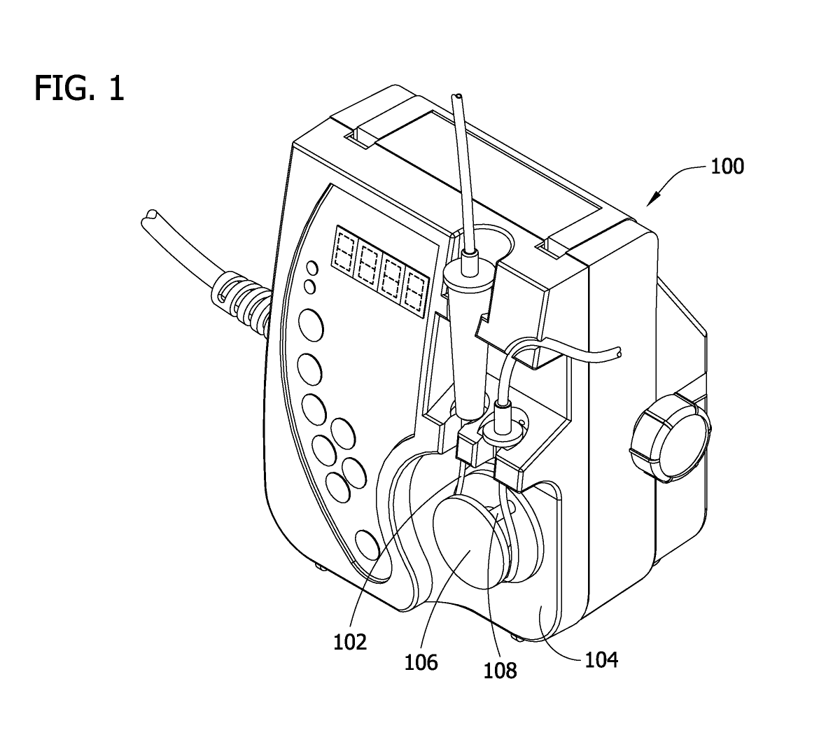

Referring to FIG. 1, a medical pumping apparatus is generally designated 100. A pump set 102 which supplies fluid from a reservoir (not shown) to a patient (not shown) is loaded on a housing 104 of the pumping apparatus 100 such that the pump set 102 is in contact with a pump rotor 106 of the pumping apparatus 100. When the pumping apparatus 100 rotates the pump rotor 106, rollers 108 occlude a portion of a tube of the pump set 102 and force the fluid from the reservoir to the patient by a peristaltic action. The pumping apparatus 100 controls rotation of pump rotor 106 to control the rate and volume of fluid delivery. Aspects of the invention permit monitoring the position and rotation of pump rotor 106 with greater accuracy and greater cost-effectiveness than conventional approaches. Embodiments of the invention achieve greater accuracy by employing sensors that are relatively unaffected by magnetic fields and produce square wave detection signals with relatively fast transition times. It is to be understood that the rotary peristaltic pump illustrated and described herein is merely exemplary and that one skilled in the art could apply aspects of the invention to other medical pumping apparatuses that employ a rotatable assembly (e.g., screw or worm drive based syringe pumps).

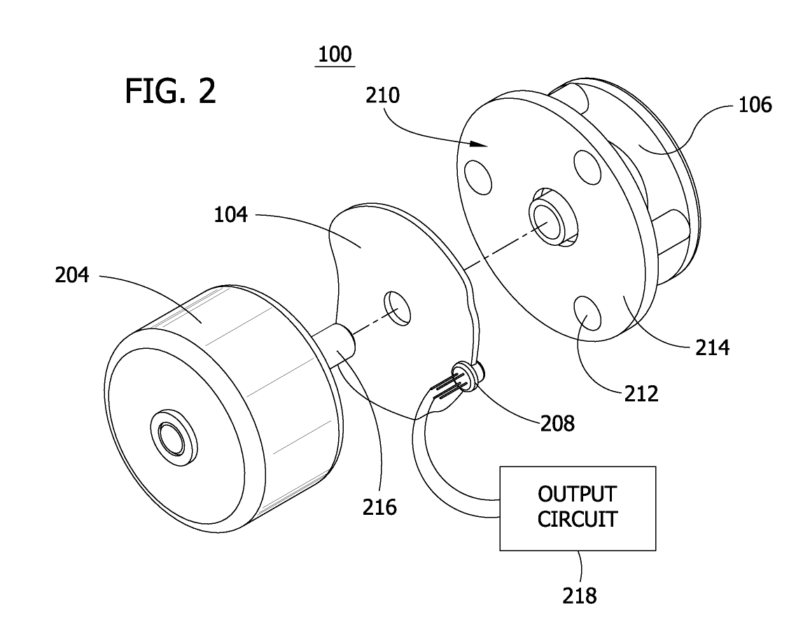

Referring to FIG. 2, the pumping apparatus 100 of FIG. 1 is shown partially exploded with a portion of the housing 104 cut away according to a first embodiment of the invention. The pumping apparatus 100 includes the housing 104, a motor 204, the pump rotor 106, and an electromagnetic radiation emitter-detector pair 208. Only the front panel of the housing 104 is shown so that the positions of the motor 204 and the emitter-detector pair 208 relative to the pump rotor 106 are visible. The electromagnetic radiation emitter-detector pair 208 emits electromagnetic radiation having a predetermined wavelength (e.g., infrared radiation or green light), and receives electromagnetic radiation including electromagnetic radiation having the predetermined wavelength. For simplicity, the electromagnetic radiation emitted by the emitter-detector pair 208 will be referred to herein as infrared radiation (IR). However, it is contemplated that the emitter-detector pair 208 may operate (i.e., emit and detect) at any wavelength and/or at multiple wavelengths without deviating from the scope of the invention.

A surface (generally designated 210) of the pump rotor 106 has a reflective portion 212 and a non-reflective portion 214. The reflective portion 212 reflects IR, and the non-reflective portion 214 absorbs, scatters, diffuses, or otherwise disperses IR such that it reflects no IR, or significantly less IR than the reflective portion 212. A shaft 216 of motor 204 passes through the housing 104 and is attached to (i.e., glued, friction fitted, fastened,, or otherwise engaged by) the pump rotor 106 at the surface 210. The shaft 216 supports the pump rotor 106 and defines its axis of rotation. When the pumping apparatus 100 supplies power to the motor 204, the motor 204 provides rotational force to the shaft 216, causing the pump rotor 106 to rotate. The emitter-detector pair 208 is mounted in the housing 104 and positioned such that as the pump rotor 106 rotates, the IR emitted by the emitter-detector pair 208 sequentially strikes the reflective portion 212 and then the non-reflective portion 214 and so forth. It is contemplated that the emitter-detector pair 208 may be mounted back from the front panel of the housing 104 and positioned to interact with the surface 210 of the pump rotor 106 through a hole, slot, or other opening in the front panel of the housing 104 without deviating from the scope of the invention.

In operation, when the non-reflective portion 214 of the pump rotor 106 is rotated through the IR emitted by the emitter-detector pair 208, the emitter-detector pair 208 receives relatively little, or no, reflected IR. Instead, the IR is scattered, absorbed, diffused, dispersed, or the like as described above. When the reflective portion 212 of the surface 210 of the pump rotor 106 rotates through the IR emitted by the emitter of emitter-detector pair 208, the detector of emitter-detector pair 208 receives a significant amount of IR (e.g., substantially equal to the amount of IR it is emitting). In one embodiment, the IR emitter-detector pair 208 generates a detection signal proportional to the amount of IR it receives. An output circuit 218, which may be part of the controller of pumping apparatus 100, receives the detection signal and compares it to a threshold. In turn, the output circuit 218 generates an output signal indicating whether the detector 208 is receiving a predetermined amount of IR reflected by the pump rotor 106 (i.e., whether the detection signal is in excess of the threshold). The threshold is a constant that is determined as a function of the construction and configuration of the pumping apparatus 100. Those skilled in the art will appreciate that the threshold is advantageously set at a level such that IR interference or noise does not cause the detection signal to exceed the threshold while still consistently indicating when the reflective portion 212 of the surface 210 of the pump rotor 106 is passing through the IR emitted by the IR emitter-detector pair 208. Thus, by monitoring the output signal for changes, a controller of the pumping apparatus 100 can determine whether the pump rotor 106 is rotating, the speed of the rotation, and the number of revolutions of the pump rotor 106 in a given period of time. Additionally, depending on the number and position of reflective 212 and non-reflective 214 portions of the surface 210 of the pump rotor 106, the output signal can be used to approximate an angular position of the pump rotor 106. The rotational speed, number of revolutions, and angular position of the pump rotor 106 may be used in any number of ways including, for example, determining a volume and rate of fluid pumped by the pumping apparatus 100.

In the pump rotor 106 of FIG. 2, the surface 210 of the pump rotor 106 has three reflective portions 212. The reflective portions 212 are spaced equal distances from the shaft 216, and are spaced equal distances from each other. The rest of the surface 210 is non-reflective such that the reflective portions 212 are separated by a single non-reflective portion 214. A controller of the pumping apparatus 100 can thus determine the angular position of the pump rotor 106 to within 120 degrees. This example is for illustration only; the pump rotor 106 may have any number of alternating reflective 212 and non-reflective 214 portions as is practical given the size of pump rotor 106.

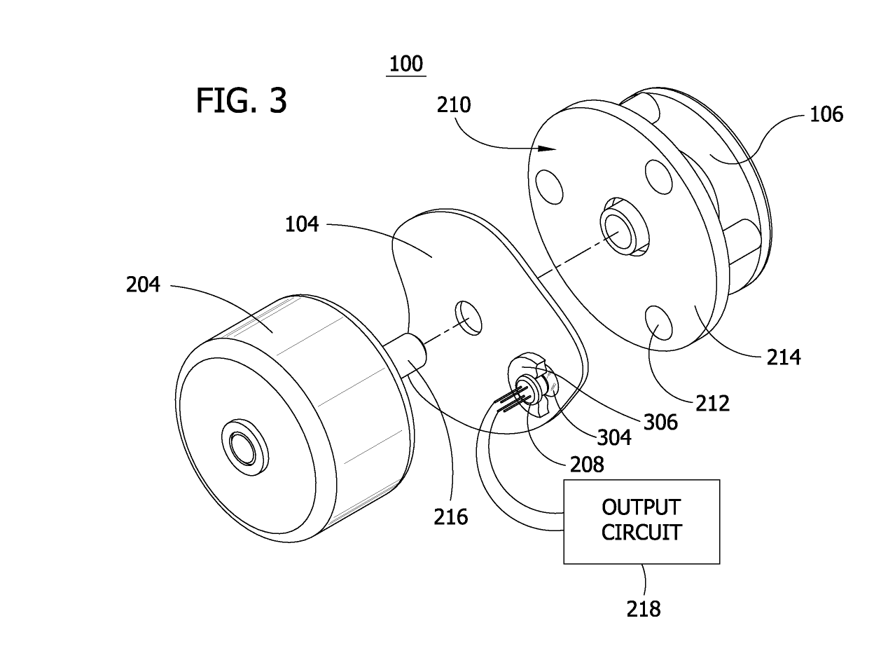

Referring now to FIG. 3, another embodiment of the pumping apparatus 100 is shown. The pumping apparatus 100 includes the motor 204, IR emitter-detector pair 208, output circuit 218, pump rotor 106, housing 104, and a transmissive window 304. The IR emitter-detector pair 208 is positioned within the housing 104 relative to the window 304 by a mount 306. In the illustrated embodiment, the mount 306 spaces the emitter-detector pair 208 back from the front panel of the housing 104. The IR emitter-detector pair 208 is positioned so that IR emitted by the emitter-detector pair 208 passes through the IR transmissive window 304 and sequentially interacts with the reflective portions 212 and the non-reflective portion 214 of the surface 210 of the pump rotor 106 as the pump rotor 106 rotates. The transmissive window 304 may be made of any material such as glass or plastic that permits IR to pass through.

The pump rotor 106 may be formed by various methods. In one embodiment, the pump rotor 106 may be injection molded using a mold that has smooth portions which correspond to the reflective portions 212, and a rough portion corresponding to the non-reflective portion 214. Alternatively, the pump rotor 106 may be machined from a block of material (e.g., plastic or aluminum) such that the pump rotor 106 has smooth portions corresponding to the reflective portions 212, and a rough (e.g., marred, hatched, scratched, or otherwise has scattering or absorptive properties relative to the electromagnetic radiation) portion corresponding to the non-reflective portion 214.

Alternatively, the pump rotor 106 may be formed (e.g., molded or machined) such that the entire surface 210 of the pump rotor 106 is reflective with respect to IR. The non-reflective portion 214 would then be added by machining (e.g., scratching or marring) the surface 210 of the pump rotor 106 to generate the non-reflective portion 214 that bounds the reflective portions 212. It is also contemplated that the pump rotor 106 may be composed of multiple pieces of material that are fastened, glued, or otherwise attached to one another to form the complete pump rotor 106.

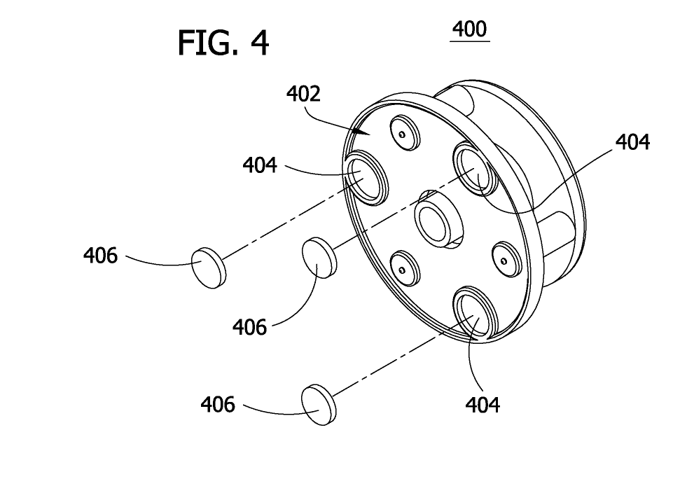

FIG. 4 illustrates a pump rotor generally designated 400 that may be used in the pumping apparatus 100 of FIGS. 1-3 in place of the pump rotor 106. The pump rotor 400 is formed such that a surface (generally designated 402) of the pump rotor 400 is non-reflective. The surface 402 has slots, recesses, or receptacles 404, for example, the receptacles required for magnets in the prior art method of pump rotor rotation detection using magnets and Hall Effect sensors. IR reflective material 406 is affixed to the pump rotor 400 in each of the receptacles 404. Alternatively, the pump rotor 400 may be formed such that the surface 402 is reflective and the material 406 affixed to the pump rotor 400 in the receptacles 404 is non-reflective without deviating from the scope of the invention. Additionally, the surface 402 may be flat and the reflective material 406 may be affixed to the surface 402 such that the receptacles 404 are not required.

It is contemplated that the reflective and non-reflective portions in the illustrated embodiments of the invention may be interchanged without deviating from the scope of the invention. It is also contemplated that the output circuit 218 may generate an output signal indicating that the non-reflective portion 214 of the pump rotor is passing through the IR emitted by the emitter 208 as opposed to indicating that the reflective portion 212 of the pump rotor 106 is passing through the IR emitted by the emitter 208, and that the output circuit 208 may be integral with the IR emitter-detector pair 208 or the controller of the medical pumping apparatus 100. Additionally, there may be any number of reflective and non-reflective portions of the pump rotor, and the reflective and non-reflective portions need not be located on the end of the pump rotor (e.g., they may spaced about the circumference of the pump rotor and the IR emitter-detector pair 208 positioned appropriately to detect the reflective and non-reflective portions). It is also contemplated that the reflective and non-reflective portions may not be evenly spaced from each other as in the illustrated embodiments of the invention, and that the emitter and detector pair 208 may operate at a frequency or wavelength other than IR. It is also contemplated that the surface of the pump rotor 106 or 400 having a reflective and non-reflective portion may be other than flat without deviating from the scope of the invention.

Embodiments of the present invention may include pumps other than rotary peristaltic pumps. For example, the present invention is applicable to syringe pumps employing screw or worm drive pumping mechanisms. In this example, one end of the worm gear is formed with a surface having reflective and non-reflective portions, and an emitter detector pair is mounted in a housing of the pump so as to interact with the reflective and non-reflective portions of the surface as the gear rotates.

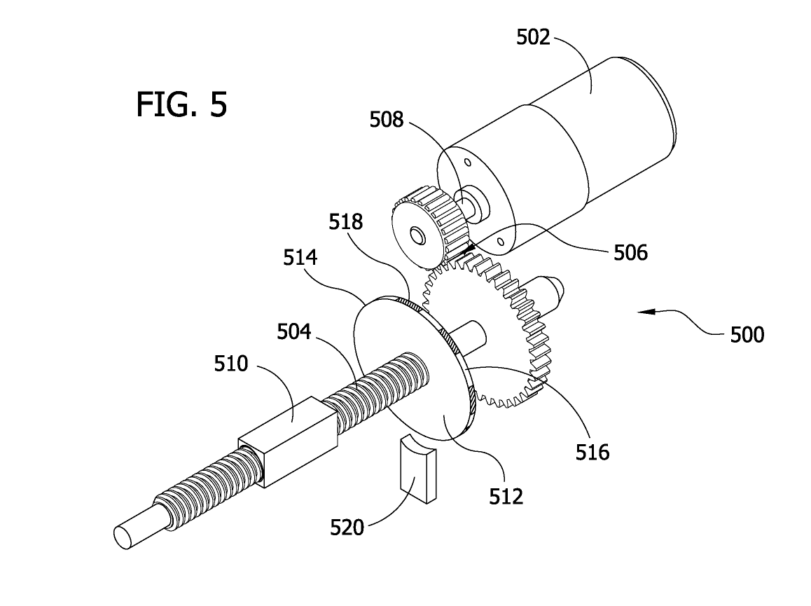

Referring now to FIG. 5, a portion of a pumping apparatus, generally designated 500, having a worm gear driven syringe pumping mechanism is shown. The pumping mechanism is driven by a motor 502, which transfers force to a worm gear shaft 504 via a gear set 506. In operation, the motor 502 drives a rotatable shaft 508 for turning the gear set 506 and, in turn, gear set 506 drives the shaft 504. As shaft 504 turns, a traveler 510 operatively connected to a syringe mechanism (not shown) moves along the length of worm gear shaft 504. A disk 512 affixed to the worm gear shaft 504 has an outer peripheral surface 514 with reflective 516 and non-reflective 518 portions that pass by an emitter detector pair 520 as the motor 502 drives the worm gear shaft 504. The emitter detector pair 520 is positioned in a plane defined by the disk 512 and directed toward the surface 514 of the disk 512. The pumping apparatus 500 thus detects rotation of the disk 512 and monitors operation of the pumping mechanism as described above.

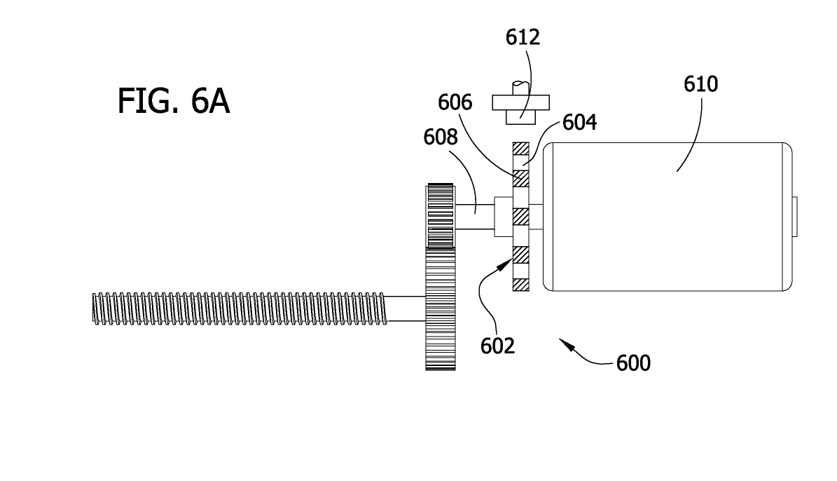

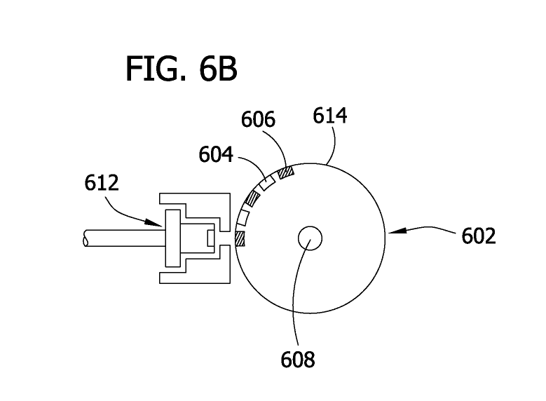

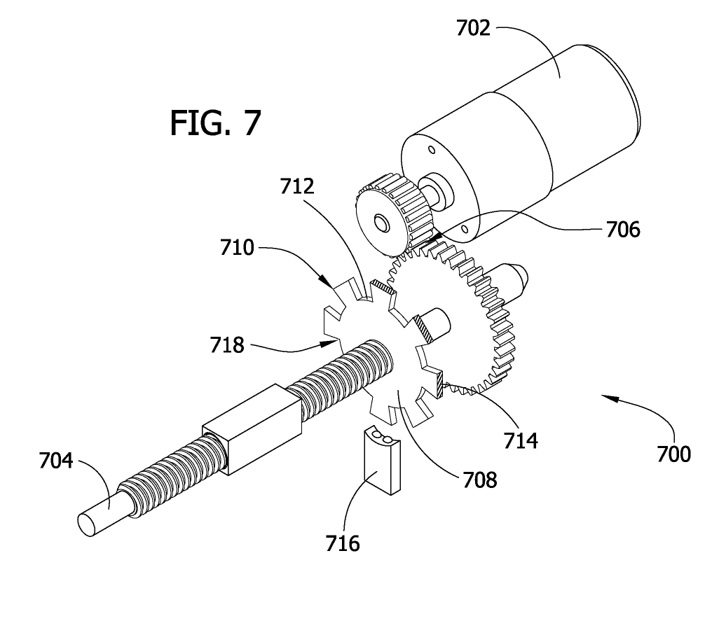

Referring now to FIGS. 6A and 6B, a portion of another embodiment of a pumping apparatus, generally designated 600, is shown. In this embodiment, a disk 602 having reflective 604 and non-reflective 606 portions on a peripheral surface 614 of the disk 602 is mounted on a shaft 608 of a motor 610 of the pumping apparatus. As the motor 610 rotates the shaft 608, the disk 602 is also rotated. The reflective 604 and non-reflective 606 portions sequentially pass in front of an emitter detector pair 612 mounted to monitor the periphery of the disk 602. That is, the emitter detector pair 612 is mounted in a plane defined by the disk 602 and directed at the disk 602. This embodiment (shown in FIGS. 6A and 6B) monitors rotation of a pumping mechanism of the pumping apparatus 600 as described above for driving, for example, a worm gear shaft of a syringe pump. [0029] Referring now to FIG. 7, a portion of a pumping apparatus, generally designated 700, having a worm gear driven syringe pumping mechanism is shown. The pumping mechanism is driven by a motor 702, which transfers force to a worm gear shaft 704 via a gear set 706. A disk 708 affixed to the worm gear shaft 704 has an outer peripheral surface 710 with non-reflective portions 714, and an inner peripheral surface 718 with reflective portions 712. Thus, the disk 708 appears to have notches where the outer surface 710 transitions to the inner surface 718. An emitter detector pair 716 is positioned in a plane defined by the disk 708 and directed toward the disk 708, such that radiation emitted by the emitter detector pair 716 interacts with the inner and outer peripheral surfaces of the disk 708. The emitter detector pair 716 is focused on the inner peripheral surface 718 such that the emitter detector pair 716 receives substantially more reflected radiation from the reflective portions 712 than the non-reflective portions 714 because the non-reflective portions 714 are out of focus with respect to the emitter detector pair 716. The pumping apparatus detects rotation of the disk 708 and monitors operation of the pumping mechanism as described above. It is contemplated that the outer peripheral surface 710 may have reflective portions 712 while the inner peripheral surface 718 has non-reflective portions 714 and the emitter detector pair 716 may be focused on the outer peripheral surface 710 without deviating from the scope of the invention.

It is to be understood that the reflective and non-reflective portions shown in FIGS. 5, 6A, 6B, and 7 may be formed in a manner similar to reflective portions 212 and non-reflective portions 214 as described above.

The order of execution or performance of the operations in embodiments of the invention illustrated and described herein is not essential, unless otherwise specified. That is, the operations may be performed in any order, unless otherwise specified, and embodiments of the invention may include additional or fewer operations than those disclosed herein. For example, it is contemplated that executing or performing a particular operation before, contemporaneously with, or after another operation is within the scope of aspects of the invention.

When introducing elements of aspects of the invention or the embodiments thereof, the articles “a,” “an,” “the,” and “said” are intended to mean that there are one or more of the elements. The terms “comprising,” “including” and “having” are intended to be inclusive and mean that there may be additional elements other than the listed elements.

Having described aspects of the invention in detail, it will be apparent that modifications and variations are possible without departing from the scope of aspects of the invention as defined in the appended claims. As various changes could be made in the above constructions, products, and methods without departing from the scope of aspects of the invention, it is intended that all matter contained in the above description and shown in the accompanying drawings shall be interpreted as illustrative and not in a limiting sense.

What is claimed is:

1. A medical pumping apparatus comprising:
a pump rotor including a surface having a reflective portion for reflecting electromagnetic radiation and a non-reflective portion that does not reflect electromagnetic radiation;
a motor for rotating the pump rotor;
an emitter for emitting electromagnetic radiation, said emitter positioned to emit electromagnetic radiation sequentially on the reflective and non-reflective portions of the surface of the pump rotor as the pump rotor rotates; and
a detector for receiving electromagnetic radiation reflected by the surface of the pump rotor and providing a detection signal indicative of the received electromagnetic radiation for monitoring rotation of the pump rotor.

2. The medical pumping apparatus of claim 1 further comprising an output circuit for receiving the detection signal and providing an output signal in response thereto, said output signal indicating whether the detector is receiving a predetermined amount of electromagnetic radiation reflected by the surface of the pump rotor.

3. The medical pumping apparatus of claim 1 wherein the emitter and detector are separated from the pump rotor by a window, said window being transmissive with respect to the electromagnetic radiation.

4. The medical pumping apparatus of claim 1 wherein:
the emitter emits infrared radiation; and
the surface of the pump rotor has a plurality of reflective portions each separated by a non-reflective portion, said reflective portions being equally spaced from each other and equally spaced from an axis about which the pump rotor rotates.

5. The medical pumping apparatus of claim 1 wherein the reflective portion of the surface of the pump rotor comprises a reflector inserted into a receptacle formed in the pump rotor.

6. The medical pumping apparatus of claim 1 wherein the reflective portion of the surface of the pump rotor comprises a smooth portion of the pump rotor produced when forming the pump rotor.

7. The medical pumping apparatus of claim 1 wherein the non-reflective portion of the surface of the pump rotor is at least one of the following: hatched, scratched, pitted, and roughed, such that said non-reflective surface diffuses electromagnetic radiation incident thereon.

8. The medical pumping apparatus of claim 1 wherein the motor comprises a shaft adapted to engage the pump rotor to transfer rotational force from the motor to the pump rotor, and wherein said shaft extends perpendicularly from the surface of the pump rotor along an axis of rotation about which the pump rotor rotates when engaged therewith.

9. A pump rotor for a medical pumping apparatus, said medical pumping apparatus having a motor, an emitter, and a detector, said pump rotor comprising:
a receiver constructed and arranged for engaging a motor shaft, said motor shaft defining an axis of rotation and supporting the pump rotor when engaged by the receiver;
a surface transverse to the axis of rotation, said surface comprising a reflective portion for reflecting electromagnetic radiation and a non-reflective portion that does not reflect electromagnetic radiation;
wherein:
the motor rotates the pump rotor via the shaft;
the emitter emits electromagnetic radiation onto the surface of the pump rotor, said emitter being positioned to sequentially emit electromagnetic radiation on the reflective and non-reflective portions of the surface of the pump rotor as the pump rotor rotates about the axis of rotation; and
the detector is positioned to receive electromagnetic radiation reflected by the surface of the pump rotor.

10. The pump rotor of claim 9 wherein the reflective portion of the surface of the pump rotor is constructed and arranged for reflecting infrared radiation emitted by the emitter.

11. The pump rotor of claim 9 wherein the surface of the pump rotor has a plurality of reflective portions each separated by a non-reflective portion, said reflective portions being equally spaced from each other and equally spaced from the axis of rotation.

12. The pump rotor of claim 9 wherein the non-reflective portion of the surface of the pump rotor is at least one of the following: hatched, scratched, pitted, and roughed, such that said non-reflective surface diffuses electromagnetic radiation incident thereon.

13. The pump rotor of claim 9 wherein the reflective portion of the surface of the pump rotor comprises a polished portion of the surface of the pump rotor.

14. The pump rotor of claim 9 wherein the reflective portion of the surface of the pump rotor comprises a reflector and further comprising a receptacle in the surface of the pump rotor sized and shaped to receive the reflector.

15. A method of detecting rotation of a rotatable assembly in a medical pumping apparatus comprising:
providing a surface on the rotatable assembly having a reflective portion for reflecting electromagnetic radiation and a non-reflective portion that does not reflect electromagnetic radiation;
emitting electromagnetic radiation onto the surface of the rotatable assembly such that as the rotatable assembly rotates, the reflective portion and the non-reflective portion of the surface of the rotatable assembly sequentially pass through the electromagnetic radiation emitted by the emitter;
receiving electromagnetic radiation reflected by the surface of the rotatable assembly at a detector of the medical pumping apparatus; and
providing a detection signal in response to receiving electromagnetic radiation reflected by the surface of the rotatable assembly at the detector.

16. The method of claim 15 further comprising receiving the detection signal and providing an output signal in response to the received detection signal, said output signal indicating whether a predetermined amount of electromagnetic radiation reflected by the surface of the rotatable assembly is being received at the detector.

17. The method of claim 15 wherein pumping apparatus comprises a housing for enclosing the emitter and the detector and further comprising passing the emitted electromagnetic radiation through a transmissive window in the housing to the surface of the rotatable assembly and passing the electromagnetic radiation reflected by the surface of the rotatable assembly back through the transmissive window to the detector.

18. The method of claim 15 wherein emitting electromagnetic radiation comprises emitting infrared radiation.

19. The method of claim 15 wherein:
providing the reflective portion of the surface comprises forming the rotatable assembly with a mold having a smooth area corresponding to the reflective portion of the surface of the rotatable assembly; and
providing the non-reflective portion of the surface of the rotatable assembly comprises at least one of:
forming the rotatable assembly with a mold having a non-smooth area corresponding to the non-reflective portion of the surface of the rotatable assembly; and
marring a portion of the surface of the rotatable assembly such that said portion diffuses the emitted electromagnetic radiation.

20. The method of claim 15 wherein:
providing the reflective portion of the surface comprises inserting a reflector into a receptacle formed in the surface of the rotatable assembly; and
providing the non-reflective portion of the surface comprises at least one of:
forming the rotatable assembly with a mold having a non-smooth area corresponding to the non-reflective portion of the surface of the rotatable assembly; and
marring a portion of the surface of the rotatable assembly such that said portion diffuses the emitted electromagnetic radiation.

21. A medical pumping apparatus comprising:
a pumping mechanism including a disk having a surface with a reflective portion for reflecting electromagnetic radiation and a non-reflective portion that does not reflect electromagnetic radiation;
a motor for rotating the pumping mechanism;
an emitter for emitting electromagnetic radiation, said emitter positioned to emit electromagnetic radiation sequentially on the reflective and non-reflective portions of the surface of the disk as the pumping mechanism rotates; and
a detector for receiving electromagnetic radiation reflected by the surface of the disk and providing a detection signal indicative of the received electromagnetic radiation for monitoring rotation of the pumping mechanism.

22. The medical pumping apparatus of claim 21 wherein the surface comprises an inner peripheral surface having the reflective portion and an outer peripheral surface having the non-reflective portion and wherein the detector is focused on the inner peripheral surface.

23. The medical pumping apparatus of claim 21 wherein the surface comprises an outer peripheral surface having the reflective portion and an inner peripheral surface having the non-reflective portion and wherein the detector is focused on the outer peripheral surface.

24. The medical pumping apparatus of claim 21 wherein the pumping mechanism is a worm gear driven syringe pump and the disk is mounted on the worm gear.

25. The medical pumping apparatus of claim 21 wherein the pumping mechanism is a worm gear driven syringe pump and the disk is mounted on a shaft of the motor, said shaft driving the worm gear.

26. The medical pumping apparatus of claim 21 wherein the surface of the disk has a plurality of reflective portions each separated by a non-reflective portion, said reflective portions being equally spaced from each other.

27. The medical pumping apparatus of claim 21 wherein the non-reflective portion of the surface of the disk is at least one of the following: hatched, scratched, pitted, and roughed, such that said non-reflective surface diffuses electromagnetic radiation incident thereon.
